(19) United States
(12) Patent Application Publication
(10) Pub. No.: US 2019/0270735 A1
(43) Pub. Date: Sep. 5, 2019

(54) Polymorphs of Dasatinib

(71) Applicant: Cipla Limited, Mumbai (IN)

(72) Inventors: Dharmaraj Ramachandra RAO, Thane (West) (IN); Geena MALHOTRA, Mumbai (IN); Srinivas Laxminarayan PATHI, Bangalore (IN); Ravikumar PUPPALA, Bangalore (IN); Yellanki JAGANNADHAM, Bangalore (IN); Ramanaiah CHENNURU, Nellore (Dt) (IN)

(21) Appl. No.: 16/344,952

(22) PCT Filed: Oct. 30, 2017
(86) PCT No.: PCT/GB2017/053257

§ 371 Date: Apr. 25, 2019

(30) Foreign Application Priority Data
Oct. 29, 2016 (IN) 201621037184
Mar. 2, 2017 (IN) 201721007409

(51) Int. Cl. C07D 417/14

(52) U.S. Cl. CPC C07D 417/14; C07B 2200/13

(57) ABSTRACT

The present invention provides novel polymorphic forms of dasatinib compound of the formula 1 as well as the process for the preparation thereof.

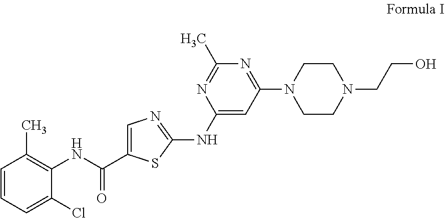 [SMILES: Cc1nc(Nc2ncc(C(=O)Nc3c(C)cccc3Cl)s2)cc(N2CCN(CCO)CC2)n1]

### CROSS REFERENCE TO RELATED APPLICATIONS

This application is a filing under 35 U.S.C. 371 of International Application No. PCT/GB2017/053257 filed Oct. 30, 2017, entitled “Polymorphs of Dasatinib” which claims priority to Indian Patent Application Nos. 201621037184 filed Oct. 29, 2016 and 201721007409 filed Mar. 2, 2017, which applications are incorporated by reference herein in their entirety.

### FIELD OF INVENTION

The present invention relates to polymorphic forms of dasatinib. In particular, the invention relates to an amorphous form and crystalline forms of dasatinib and processes for the preparation thereof.

### BACKGROUND OF THE INVENTION

Dasatinib is an oral Bcr-Abl tyrosine kinase inhibitor and marketed by Bristol-Myers Squibb under the trade name Sprycel®. It is chemically described as, N-(2-chloro-6-methylphenyl)-2-[[6-[4-(2-hydroxyethyl)-1-piperazinyl]-2-methyl-4-pyrimidinyl]amino]-5-thiazolecarboxamide and structurally represented as formula I,

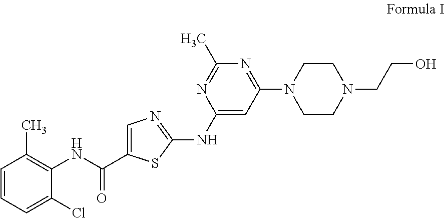 [SMILES: Cc1nc(Nc2ncc(C(=O)Nc3c(C)cccc3Cl)s2)cc(N2CCN(CCO)CC2)n1]

U.S. Pat. No. 6,596,746 discloses an analogous process for the preparation of dasatinib by reacting a compound of N-(2-hydroxyethyl)piperizine with appropriate amine of formula II at a temperature of 80° C. for 2 hours.

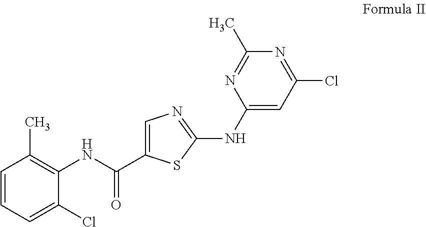 [SMILES: Cc1nc(Cl)cc(Nc2ncc(C(=O)Nc3c(C)cccc3Cl)s2)n1]

U.S. Pat. No. 7,491,725 discloses different polymorphic forms of dasatinib, such as crystalline monohydrate form, crystalline neat form and the solvates of dasatinib, such as butanol, ethanol, diethanol solvates. The patent discloses the characterization of these polymorphs by X-ray diffraction technique and differential scanning calorimetry.

U.S. Pat. No. 7,973,045 describes the preparation of a number of solvates or mixed solvates of dasatinib, like an n-propanol-dimethylsulfoxide solvate, a DMSO solvate, a hemi-tetrahydrofuran solvate, a 2-methyl-tetrahydrofuran solvate, a hemi 1,4-dioxane solvate, a pyridine solvate, a toluene solvate, a methyl isobutyl ketone solvate, a mono-acetone solvate, an iso-propanol-DMSO solvate, a 2-butanol-DMSO solvate, an IPA-DMF solvate, an IPA solvate, an n-propanol-DMF solvate, an n-propanol solvate, a 2-butanol-DMF solvate, a 2-butanol solvate, an n-butanol-DMSO solvate, a DMF-water solvate, a DMF solvate, a methyl isopropyl ketone solvate, a dimethoxyethane solvate, a cellosolve solvate, a methylacetate solvate, a methanol solvate, an ethylacetate solvate, a 2-pentanole solvate, a dimethyl carbonate solvate, an isopropylacetate solvate, a ethyleneglycol solvate, a dichloromethane solvate, a methylformate solvate, a tert-butanol solvate, a dimethoxyethane solvate, a methylethylketone (“MEK”) solvate, a monochlorobenzene solvate, a propylene glycol monoethyl ether (“PGME”) solvate, a glycerol solvate, a cyclopentyl methyl ether solvate, a methyl tert-butyl ether (“MTBE”) solvate, an amylalcohol solvate, and a glycerol formal solvate.

WO 2010/062715A2 discloses isosorbide dimethyl ether solvate, N,N′-dimethylethylene urea solvate and N,N′-dimethyl-N,N′-propylene urea solvate of Dasatinib.

WO 2012/014149 describes a crystalline N-methylformamide solvate of dasatinib.

US 2016/0264565 describes a crystalline Form-SDI of Dasatinib, wherein the crystalline Form-SDI is characterised by 3-methylbutan-1-ol content in range of 10-16% w/w.

WO 2017/002131 describes a crystalline 1,2-propanediol solvate of dasatinib, wherein 1,2-propanediol content is not more than 15%.

WO 2015/049645 discloses an amorphous dihydrate form of dasatinib, an amorphous monohydrate form of dasatinib and an amorphous hemihydrate form of dasatinib. The amorphous dihydrate form contains about 5% to 9% of moisture content, the amorphous monohydrate form contains about 3% to 5% of moisture content and the amorphous hemihydrate contains 0.5% to 3% of moisture content. The patent discloses the characterization of these polymorphs by X-ray diffraction technique.

In view of the above discussed prior art references, it is evident that dasatinib exhibits different polymorphic forms under differential conditions that include solvent, moisture, temperature, time and drying conditions and thus the bioavailability of the same also varies with polymorphic modification.

Polymorphism is the ability of a solid material to exist in two or more forms or crystalline structures of the same chemical compound and it is a solid-state phenomenon. Polymorphism essentially means that in different polymorphs, the same type of molecules exist in different ways. If such types of differences exist due to its packing it is termed as packing polymorphism and if it exists due to its difference in conformation it is termed as conformational polymorphism.

Molecules of crystals have different arrangement in the unit cell, as a result of polymorphism and thus display different physical properties like packing properties, spectroscopic properties, thermodynamic properties such as free energy, solubility, melting point, etc., kinetic properties such as rate of dissolution, stability, and mechanical properties such as compatibility, hardness, tensile strength, etc.

A change in crystal structure of compound by means of change in polymorphism affects physicochemical properties like dissolution and solubility, chemical and physical stability, flowability and hygroscopicity of a compound. Therefore, there remains a need in the art for polymorphic form of dasatinib which is having greater stability, flowability, dissolution properties; thereby increasing the bioavailability of the drug.

An amorphous form generally provides better solubility and bioavailability than the crystalline form and may be useful for formulations which can have better stability, solubility and compressibility etc. which are important for formulation and product manufacturing.

Therefore, it is desirable to have a stable amorphous form of drug with high purity to meet the needs of regulatory agencies and highly reproducible processes for its preparation.

### OBJECTIVES OF THE INVENTION

It is an object of the present invention to provide novel forms of dasatinib having advantageous properties. It is also an object of the present invention to provide processes for preparing the novel forms and pharmaceutical formulations incorporating the novel forms.

### BRIEF DESCRIPTION OF DRAWINGS

FIG. 1 depicts an X-ray diffraction spectrum of amorphous form of dasatinib.

FIG. 2 depicts differential scanning calorimetry (DSC) curve of amorphous form of dasatinib.

FIG. 3 depicts an X-ray powder diffraction (XRPD) of crystalline Form C2 of dasatinib.

FIG. 4 depicts an X-ray powder diffraction (XRPD) of crystalline Form C3 of dasatinib.

FIG. 5 depicts an X-ray powder diffraction (XRPD) of crystalline Form C4 of dasatinib.

### SUMMARY OF THE INVENTION

The present invention provides novel polymorphic forms of dasatinib which exhibit enhanced solubility in water, permeability, bioavailability and be suitable for bulk handling and formulation.

The present invention provides an amorphous form of dasatinib having a glass transition temperature of about 153° C.±3° C.

According to the second aspect of the invention, there is provided novel crystalline solvates of dasatinib. The present invention provides a crystalline polymorph of dasatinib, wherein the polymorph is a mono benzyl alcohol solvate. The said crystalline solvates are referred to as Form C2 and Form C3. Each of the new crystalline solvates is differentiated by a unique powder X-ray diffraction pattern.

Accordingly, the invention also provides methods for preparing these novel amorphous and crystalline forms in high purity and high yield.

In accordance with yet another object, the present invention provides a pharmaceutical composition comprising an amorphous form of dasatinib having a glass transition temperature of about 153° C.±3° C. in combination with one or more pharmaceutically acceptable excipients.

In accordance with yet another object, the present invention provides a pharmaceutical composition comprising a novel crystalline solvate of dasatinib as described herein in combination with one or more pharmaceutically acceptable excipients.

### DETAILED DESCRIPTION OF THE INVENTION

The invention will now be described in detail in connection with certain preferred and optional aspects, so that various features thereof may be more fully understood and appreciated.

According to the first aspect, the present invention provides an amorphous form of dasatinib.

An amorphous form of dasatinib is characterised by a differential scanning calorimetry curve that comprises a glass transition temperature of about 153° C.±3° C.

The Differential Scanning Calorimetry (DSC) experiments were performed on a TA Waters Discovery DSC. The following experimental conditions were used:

| | | | |
|---|---|---|---|
| 1. | Isothermal | 5 | min |
| 2. | Rate of heating | 2° | C./min |
| 3. | N2 Flow | 50 | ml/min |
| 4. | Modulation temperature | 0.7° | C./60 s |

| | | |
|---|---|---|
| 5. | Heating range | 0° C.-200° C. |
| 6. | Pan Type | Tzero Alluminium |

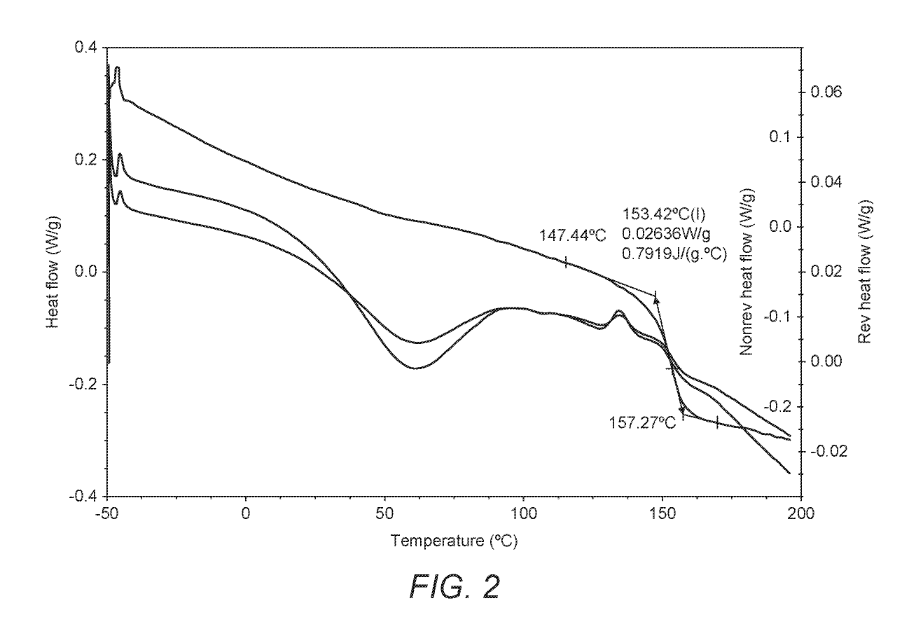

The DSC experiments were used to ascertain the glass transition temperature of the amorphous form. FIG. 2 shows a glass transition temperature of 153.42° C. The amorphous form of the present invention may be characterized as having a glass transition temperature of 153° C.±3° C.

In some embodiments, the amorphous form is further characterized by using various techniques including, but not limited to, polarized light microscopy (PLM), thermal analysis (e.g. thermogravimetric analysis (TGA) and Infrared spectrum (IR).

The present invention also provides a process for the preparation of an amorphous form of dasatinib comprising the steps of:
a) mixing dasatinib in a solvent or a mixture of solvents
b) treating the mixture from step a) with an acid,
c) heating the mixture from step b) to reflux temperature, and
d) treating the mixture from step c) with a base to isolate amorphous dasatinib.

The present invention also provides an amorphous form of dasatinib obtained by the process mentioned above.

The solvent used in step a) of the process may be selected from the group consisting of polar solvents or a mixture of polar solvents. Polar solvents may be selected from, but not limited to water, alcohol and acetone or any combination thereof. The alcohol may be selected from, but not limited to methanol, ethanol, isopropyl alcohol, n-butanol and 2-butanol or any combination thereof. Preferably, the solvent is methanol or n-butanol.

Dasatinib used in step a) can be prepared by any method as disclosed in the prior art. Further, dasatinib used could be in any form, such as crystalline, hydrates and solvates.

The acid used in step b) of the process is selected from an inorganic acid or an organic acid. The inorganic acid used in step b) is selected from, but not limited to hydrochloric acid, boric acid, phosphoric acid or any combination thereof. The organic acid used in step b) is selected from, but not limited to formic acid, acetic acid, oxalic acid, citric acid, succinic acid, benzoic acid, p-toluenesulfonic acid or any combination thereof. Preferably, the acid is phosphoric acid.

In step c) of the process, the reaction mixture is heated at a reflux temperature of the solvent. The reaction mixture obtained in step c) of the process is heated at a reflux temperature ranging from 50° C. to 95° C. Preferably, the temperature is 50° C. to 60° C.

The reaction mixture obtained in step c) of the process is heated at reflux temperature ranging from 0.5 hour to 4 hours. Preferably, 1 hour. Furthermore, the reaction mixture obtained in step c) of the process is optionally cooled to a temperature range of 15° C. to 45° C. Preferably, the temperature is 25° C. to 35° C.

The mixture obtained in step c) of the process may be filtered and treated with water. The mixture of step c) can also be directly used in subsequent step d), without dilution with water.

In step d) of the process, the mixture obtained in step c) is treated with a base.

The base used in step d) of the process may be selected from an inorganic base or an organic base. The said base is selected from, but not limited to, ammonia, triethylamine, potassium carbonate, potassium bicarbonate, sodium carbonate and sodium bicarbonate or any combination thereof. Preferably, the base is ammonia.

The base used in step d) is added to the reaction mixture at a temperature range of 5° C. to 30° C. Preferably the temperature is 15° C. to 20° C.

The reaction mixture in step d) of the process is stirred for 0.5 hour to 3 hours to isolate amorphous form of dasatinib. Preferably, the reaction mixture is stirred for 1 hour.

The presently claimed process results in an isolated amorphous form of dasatinib.

The isolated amorphous form of dasatinib may be characterised by having glass transition temperature of about 153° C.±3° C.

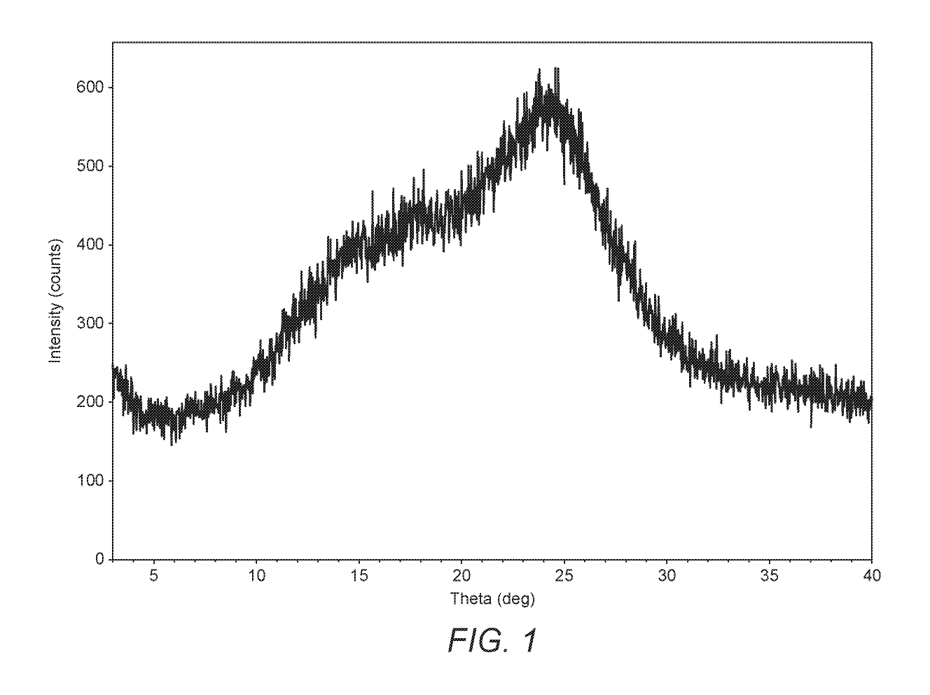

An amorphous form of dasatinib is characterised by a differential scanning calorimetry curve depicted in FIG. 2. An amorphous form of dasatinib may be further characterised by X-ray diffraction (XRD) as depicted in FIG. 1.

X-Ray Powder diffraction was carried out in a Rigaku D-Max 2200 X-Ray diffractometer. The D-Max 2200 system was equipped with a 1.2 kW Cu anode X-ray tube and a scintillation detector. Cu K alpha radiation (λ=1.5405 Å) was used to obtain all patterns. Nickel filter was placed in the receiving path of the X-ray to remove Cu Kβ radiation. Samples were ground using mortar and pestle to reduce the crystal size and orientation effects. Material was then tightly packed in a glass holder using glass slides to match the surface level of the sample holder and analyzed using the following parameters.

| I. | Source: | Cu Kα | |
|---|---|---|---|
| II. | Wavelength: | ~1.5405 | A° |
| III. | Voltage: | 40 | KV |
| IV. | Current: | 30 | mA |
| V. | Scan axis: | theta/2 theta | |
| VI. | Measurement method: | Continuous | |
| VII. | Scanning range: | 3°-40° 2θ | |
| VIII. | Goniometer speed: | 2° 2θ/min | |
| IX. | Sampling width: | 0.02° 2θ | |
| X. | Divergence slit: | 1° | |
| XI. | Receiving slit: | 0.3 | mm |

| | | |
|---|---|---|
| XII. | Counting unit: | cps |
| XIII. | Detector type: | Scintillation counter |

The present invention also provides an alternative process for the preparation of amorphous form of dasatinib.

In one aspect of the invention, the process for preparation of an amorphous form of dasatinib comprising the steps of:
a) mixing dasatinib with a solvent or a mixture of solvents thereof,
b) heating the reaction mixture at a temperature of 50° C. to 90° C.,
c) treating the reaction mixture with at least one water miscible solvent or a mixture thereof, and
d) isolating the amorphous form of dasatinib.

The solvent used in step a) of the process may be selected from the group consisting of polar solvents or a mixture of polar solvents. Polar solvents may be selected from, but not limited to water, alcohol and acetone or any combination thereof. The alcohol may be selected from, but not limited to methanol, ethanol, isopropyl alcohol, n-butanol and 2-butanol or any combination thereof. Preferably, the solvent is methanol or n-butanol.

The water miscible solvent may be selected from the group comprising acetonitrile, acetone, DMF, DMSO and THF or any combination thereof.

In another aspect, an amorphous form of dasatinib is prepared without isolation of dasatinib from the reaction mixture in any polymorphic form.

The process of the invention is advantages as it produces amorphous form of dasatinib characterised by having glass transition temperature of about 153° C.±3° C., that has less than about 10%, preferably less than about 5%, more preferably less than about 1%, and most preferably does not contain any detectable crystalline form of dasatinib.

The present invention also provides, the use of an amorphous form of dasatinib in method for treating cancer comprising administering to a patient in need thereof a therapeutically effective amount of dasatinib.

Dasatinib can be prepared by any method as disclosed in the prior art.

Dasatinib can be prepared by condensation of 2-((6-chloro-2-methylpyrimidin-4-yl)amino)-N-(2-chloro-6-methylphenyl)thiazole-5-carboxamide with N-(2-hydroxylethyl) piperazine in the presence of a solvent.

The advantages of the process include simplicity of manufacturing, eco-friendliness and suitability for commercial use.

In a further aspect, the present invention provides a pharmaceutical composition comprising an amorphous form of dasatinib as described herein. More specifically, there is provided an amorphous form of dasatinib having a glass transition temperature of about 153° C.±3° C. and one or more of pharmaceutically acceptable carriers, excipients or diluents for use in the treatment of cancer.

Dasatinib obtained by the present invention may be amorphous in nature and stable. The amorphous forms are generally readily soluble than their crystalline counter parts and therefore, the amorphous form of dasatinib provided according to the invention is expected to have higher dissolution, solubility and hence bioavailability.

In a further aspect, the amorphous dasatinib obtained by the processes disclosed in the present invention may be formulated as solid compositions for oral administration in the form of capsules, tablets, pills, powders or granules useful in treating cancer.

The present invention further provides crystalline solvates of dasatinib. As used herein, the term “solvate” means the formation of a crystalline complex of variable stoichiometry comprising (in this invention), a compound of Formula (I) and a solvent. The term “solvate” shall be interpreted accordingly.

According to the second aspect of the invention, the present invention provides a crystalline polymorph of dasatinib, wherein the polymorph is a mono benzyl alcohol solvate. The present invention also provides a crystalline Form C2 mono benzyl alcohol solvate of dasatinib.

In an aspect of the present invention, the crystalline Form C2 of dasatinib exhibits peaks at two or more angles selected from the group consisting of 3.62, 5.50, 11.20, 16.920 (±0.2020).

The crystalline Form C2 of dasatinib exhibits further peaks selecting from the group consisting of 22.24 and 24.54° as a diffraction angle (±0.2° 2θ) in X-ray powder diffraction.

In another aspect of the present invention, the crystalline Form C2 of dasatinib exhibits peaks at two or more angles selected from the group consisting of 12.26, 13.66, 14.78, 17.48 and 18.18° as a diffraction angle (±0.2° 2θ) in X-ray powder diffraction.

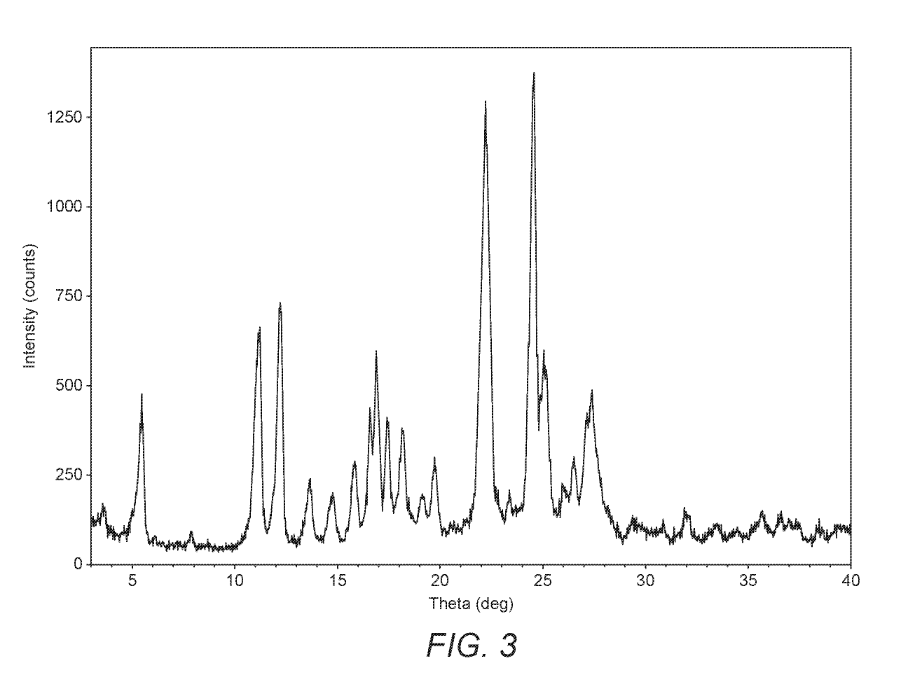

In yet another aspect of the present invention, the crystalline Form C2 of dasatinib is characterised by X-ray powder diffraction as illustrated in FIG. 3.

The present invention provides a process for the preparation of crystalline Form C2 of dasatinib comprising the steps of:
i) treating dasatinib with benzyl alcohol and optionally one or more further solvents,
ii) optionally heating the mixture at a temperature range of 20° C. to 70° C., and
iii) isolating crystalline Form C2 of dasatinib.

The present invention also provides a crystalline Form C2 of dasatinib obtained by the process mentioned above.

Dasatinib used in step i) can be prepared by any method as disclosed in the prior art. Further, dasatinib can be used in any form, such as amorphous, crystalline, hydrates or solvates.

In one aspect of the present invention, the or each further solvent used in step (i) of the process is an alcohol or a mixture of alcohols (other than benzyl alcohol). The alcohol may be selected from, but not limited to methanol, ethanol, isopropyl alcohol, n-butanol, 2-butanol, 2-phenylethyl alcohol, 2-phenoxyethanol, 3-phenoxypropanol, 1-phenoxy-propan-2-ol, 3-phenoxy-propane-1,2-diol, and benzyloxymethanol or mixtures thereof. The alcohol may be methanol or ethanol or any combination thereof. Suitably, the solvent used in step (i) is benzyl alcohol alone, i.e. without the presence of a further solvent.

In step (i) of the process dasatinib can be treated with a solvent at room temperature.

In step (ii) of the process dasatinib can be treated with a solvent at a temperature range of 20° C. to 70° C. Preferably, the temperature is 25° C.

The mixture obtained in step (i) or step (ii) of the process is stirred for 10 to 48 hours to yield a crystalline mixture. Preferably, 24 to 48 hours.

In step (iii) of the process the crystalline mixture is filtered and dried to obtain crystalline Form C2 of dasatinib.

The crystalline mixture can be dried in vacuum tray dryer at a temperature range of 30° C. to 60° C.

In another aspect, a crystalline Form C2 of dasatinib is prepared by isolation of dasatinib from the reaction mixture. In another aspect, a crystalline Form C2 of dasatinib is prepared without isolation of dasatinib base from the reaction mixture in any polymorphic form. Dasatinib can be prepared by any method as disclosed in the prior art.

Dasatinib can be prepared by condensation of 2-((6-chloro-2-methylpyrimidin-4-yl)amino)-N-(2-chloro-6-methylphenyl)thiazole-5-carboxamide with N-(2-hydroxylethyl) piperazine in the presence of a solvent.

The present invention also provides a pharmaceutical composition comprising a crystalline Form C2 of dasatinib and one or more of pharmaceutically acceptable carriers, excipients or diluents for use in the treatment of cancer.

The crystalline Form C2 of dasatinib of the present invention may be formulated as solid compositions for oral administration in the form of capsules, tablets, pills, powders or granules.

The present invention also provides, the use of crystalline Form C2 of dasatinib in a method for treating cancer comprising administering to a patient in need thereof a therapeutically effective amount of dasatinib.

According to the third aspect of the invention, the present invention provides a crystalline Form C3 of dasatinib. The Form C3 is crystalline mono benzyl alcohol solvate of dasatinib.

In one aspect of the present invention, the crystalline Form C3 of dasatinib exhibits peaks at two or more angles selected from the group consisting of 5.48, 11.00, 12.20, 16.56, 16.860 (±0.2° 2θ). The crystalline Form C2 of dasatinib exhibits further peaks selecting from the group consisting of 18.12 and 24.48° as a diffraction angle (±0.2° 2θ) in X-ray powder diffraction.

In another aspect of the present invention, the crystalline Form C3 of dasatinib exhibits peaks at two or more angles selected from the group consisting of 13.62, 14.72, 19.68 and 22.18° as a diffraction angle (±0.2° 2θ) in X-ray powder diffraction.

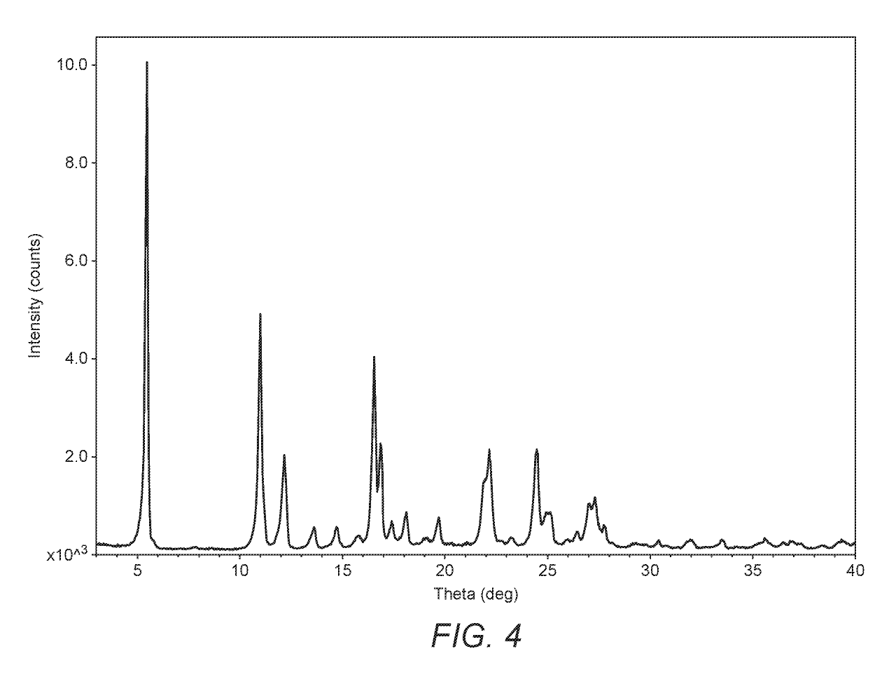

In yet another aspect of the present invention, the crystalline Form C3 of dasatinib is characterised by X-ray powder diffraction as illustrated in FIG. 4.

The present invention also provides a process for the preparation of crystalline Form C3 of dasatinib comprising the steps of:
i) treating dasatinib with benzyl alcohol and optionally one or more further solvents,
ii) optionally heating the mixture at a temperature range of 20° C. to 70° C., and
iii) isolating crystalline Form C3 of dasatinib.

The present invention also provides a crystalline Form C3 of dasatinib obtained by the process mentioned above.

Dasatinib used in step (i) can be prepared by any method as disclosed in the prior art. Further, dasatinib can be used in any form, such as amorphous, crystalline, hydrates or solvates.

In an aspect of the present invention, the solvent used in step (i) of the process is selected from the group consisting of alcohols, hydrocarbons and ethers or a mixture thereof.

The or each further alcohol (i.e. other than benzyl alcohol) is selected from, but not limited to methanol, ethanol, isopropyl alcohol, n-butanol, 2-butanol, 2-phenylethyl alcohol, 2-phenoxyethanol, 3-phenoxypropanol, 1-phenoxy-propan-2-ol, 3-phenoxy-propane-1,2-diol, and benzyloxymethanol or mixtures thereof. More particularly, the further alcohol may be methanol or ethanol or a mixture thereof.

The hydrocarbon may be selected from, but not limited to benzene, hexane, cyclohexane, cyclohexene, heptane, cycloheptane, xylene and toluene or mixtures thereof.

The ether may be selected from, but not limited to diisopropyl ether, dimethoxyethane, dimethoxymethane 1,4-dioxane, di-tert-butyl ether, diethyl ether, glycol ethers, diethylene glycol, diethyl ether, ethyl tert-butyl ether, methyl tert-butyl ether and tetrahydrofuran or mixtures thereof.

Suitably, the solvent used in step (i) is a mixture of benzyl alcohol and methanol. Alternatively, the solvent used in step (i) is a mixture of benzyl alcohol and ethanol.

In the step (i) of the process dasatinib can be treated with a solvent or mixtures thereof at room temperature. In step (ii) of the process dasatinib can be treated with a solvent or mixtures thereof at a temperature range of 20° C. to 70° C. Preferably, the temperature is 25° C. or 40° C.

The mixture obtained in step (i) or step (ii) of the process is stirred for 10 to 48 hours to yield crystalline mixture. Preferably, 15 to 24 hours.

In an aspect of the present invention, in the step (iii) of the process the crystalline mixture is filtered and dried to yield crystalline Form C3 of dasatinib.

In another aspect of the present invention, the crystalline mixture can be dried in vacuum tray dryer at a temperature range of 30° C. to 60° C.

In another aspect, a crystalline Form C3 of dasatinib is prepared by isolation of dasatinib from the reaction mixture. In another aspect, a crystalline Form C3 is prepared without isolation of dasatinib from the reaction mixture in any polymorphic form. Dasatinib can be prepared by any method as disclosed in the prior art.

Dasatinib can be prepared by condensation of 2-((6-chloro-2-methylpyrimidin-4-yl)amino)-N-(2-chloro-6-methylphenyl)thiazole-5-carboxamide with N-(2-hydroxylethyl) piperazine in the presence of a solvent.

The present invention also provides a pharmaceutical composition comprising a crystalline Form C3 of dasatinib and one or more of pharmaceutically acceptable carriers, excipients or diluents for use in the treatment of cancer.

The crystalline Form C3 of dasatinib of the present invention may be formulated as solid compositions for oral administration in the form of capsules, tablets, pills, powders or granules.

The present invention also provides, the use of crystalline Form C3 of dasatinib in a method for treating cancer comprising administering to a patient in need thereof a therapeutically effective amount of dasatinib.

According to the fourth aspect of the invention, the present invention provides a crystalline Form C4 of dasatinib. In another aspect, the Form C4 is crystalline mono glycerol solvate of dasatinib.

In an aspect of the present invention, the crystalline Form C4 of dasatinib exhibiting peaks at two or more angles selected from the group consisting of 7.04, 11.80, 14.16, 15.90 and 24.02° as a diffraction angle (±0.2° 2θ) in X-ray powder diffraction.

In an aspect of the present invention, the crystalline Form C4 of dasatinib exhibiting peaks at two or more angles selected from the group consisting of 18.54, 18.98, 20.60 and 24.90° as a diffraction angle (±0.2° 2θ) in X-ray powder diffraction.

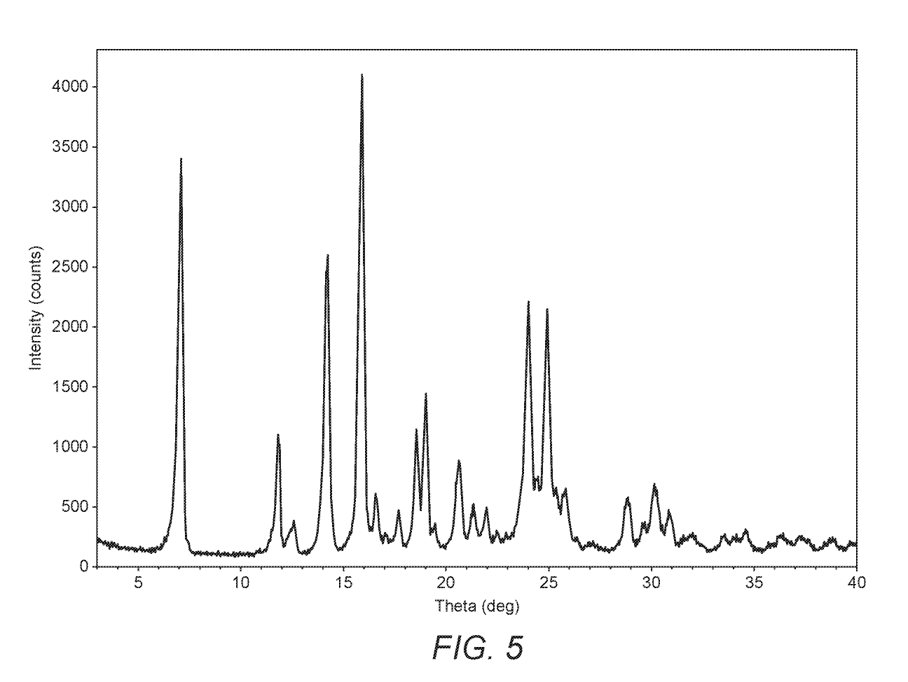

In another aspect of the present invention, the crystalline Form C4 of dasatinib is characterised by X-ray powder diffraction as illustrated in FIG. 5.

According to an aspect of the invention, the present invention provides a process for the preparation of crystalline Form C4 of dasatinib comprising the steps of:
1) treating dasatinib with a solvent or a mixture thereof,
2) optionally heating the mixture at a temperature range of 30° C. to 70° C., and
3) isolating crystalline Form C4 of dasatinib.

In an aspect, dasatinib used in step (1) can be prepared by any method as disclosed in the prior art. Further, dasatinib can be used in any form, such as amorphous, crystalline, hydrates or solvates.

In an aspect of the present invention, the solvent used in step (1) of the process is selected from the group consisting of polyol and alcohol or a mixture thereof.

The polyol is selected from sugar alcohols. The polyol is selected from but not limited to glycol, glycerol, erythritol, threitol, arabitol, xylitol, ribitol, mannitol and sorbitol.

The alcohol is selected from, but not limited to methanol, ethanol, isopropyl alcohol, n-butanol, 2-butanol and benzyl alcohol or mixtures thereof.

In an aspect of the present invention, in the step (1) of the process dasatinib can be treated with a solvent or mixtures thereof at room temperature.

In an aspect of the present invention, in the step (2) of the process dasatinib can be treated with a solvent or mixtures thereof at a temperature range of 30° C. to 70° C.

The mixture obtained in step (1) or step (2) of the process is stirred for 10 to 48 hours to yield crystalline mixture.

In an aspect of the present invention, in the step (3) of the process the crystalline mixture is filtered and dried to obtain crystalline Form C4 of dasatinib.

In another aspect of the present invention, the crystalline mixture can be dried in vacuum tray dryer at a temperature range of 30° C. to 60° C.

In another aspect, a crystalline Form C4 of dasatinib is prepared without isolation of dasatinib from the reaction mixture in any polymorphic form. Dasatinib can be prepared by any method as disclosed in the prior art.

Dasatinib can be prepared by condensation of 2-((6-chloro-2-methylpyrimidin-4-yl)amino)-N-(2-chloro-6-methylphenyl)thiazole-5-carboxamide with N-(2-hydroxylethyl) piperazine in the presence of a solvent.

In an aspect, the present invention provides a pharmaceutical composition comprising a crystalline Form C4 of dasatinib and one or more of pharmaceutically acceptable carriers, excipients or diluents for use in the treatment of cancer.

The crystalline Form C4 of dasatinib of the present invention may be formulated as solid compositions for oral administration in the form of capsules, tablets, pills, powders or granules.

The following examples, which include preferred aspects, will serve to illustrate the practice of this invention, it being understood that the particulars shown are by way of example and for purpose of illustrative discussion of preferred aspects of the invention.

### EXAMPLES

### Example 1—Amorphous Dasatinib

To a reaction flask, was charged dasatinib (20 g), methanol (200 mL) and phosphoric acid (10 mL) and the reaction mixture was refluxed at a temperature of 50° C. to 60° C. The reaction mixture was stirred for 1 hour at a temperature of 50° C. to 60° C. The mixture obtained was then cooled to 25° C. to 35° C. and stirred for 1 hour maintaining the temperature. The resulting reaction mixture was then filtered and washed with methanol. To the reaction mixture water (400 mL) was then charged and further cooled at a temperature of 15° C. to 20° C. Then liquid ammonia (20 mL) was slowly added to the reaction mixture maintaining the temperature at 15° C. to 20° C. The reaction mixture was further stirred for 1 hour to isolate the product, amorphous dasatinib. The product was then filtered, washed with water and dried under vacuum.

### Example 2—Amorphous Dasatinib

To a reaction flask was charged dasatinib and dimethylsulfoxide and the reaction mixture was heated at a temperature of 70° C. to 80° C. with stirring. In another reaction flask, charged water was cooled to a temperature of 5° C. to 10° C. To the cooled water, the charged reaction mixture containing dasatinib was added and the mixture was stirred for 1 hour. The precipitated product was filtered, leaving amorphous dasatinib.

### Example 3—Amorphous Dasatinib

To a reaction flask, charged dasatinib crystalline neat form (N-6) (20 g), methanol (200 mL) and phosphoric acid (10 mL) were added and the reaction mixture was refluxed at a temperature of 50° C. to 60° C. The reaction mixture was stirred for 1 hour at a temperature of 50° C. to 60° C. The mixture obtained was then cooled to 25° C. to 35° C. and stirred for 1 hour maintaining the temperature. The resulting reaction mixture was then filtered and washed with methanol. To the reaction mixture, charged water (400 mL) was added and further cooled at a temperature of 15° C. to 20° C. Liquid ammonia (20 mL) was slowly added to the reaction mixture, maintaining the temperature at 15° C. to 20° C. The reaction mixture was further stirred for 1 hour to isolate the product, amorphous dasatinib. The product was filtered, washed with water and dried under vacuum.

### Example 4—Amorphous Dasatinib

To a reaction flask, charged dasatinib crystalline neat form (N-6) and dimethylformamide were added and heated at a temperature of 70° C. to 80° C. with stirring. In another reaction flask, charged water was added and cooled at a temperature of 5° C. to 10° C. To this cooled water, the charged reaction mixture containing dasatinib was added and stirred for 1 hour. The precipitated product was filtered, leaving amorphous dasatinib.

### Example 5—Amorphous Dasatinib

To a reaction flask charged dasatinib crystalline monohydrate (20 g), methanol (200 mL) and phosphoric acid (10 mL) were added and the reaction mixture was refluxed at a temperature of 50° C. to 60° C. The reaction mixture was stirred for 1 hour at temperature of 50° C. to 60° C. The mixture obtained was then cooled to 25° C. to 35° C. and stirred for 1 hour maintaining the temperature. The resulting reaction mixture was then filtered and washed with methanol. To the reaction mixture, the charged water (400 mL) was added and further cooled at a temperature of 15° C. to 20° C. Liquid ammonia (20 mL) was slowly added to the reaction mixture maintaining the temperature at 15° C. to 20° C. The reaction mixture was stirred for 1 hour to isolate the product, amorphous dasatinib. The product was filtered, washed with water and dried under vacuum.

### Example 6—Amorphous Dasatinib

To a reaction flask, charged dasatinib crystalline monohydrate and dimethylsulfoxide were added and the reaction mixture was heated at a temperature of 70° C. to 80° C. with stirring. In another reaction flask, charged water was cooled at a temperature of 5° C. to 10° C. To this cooled water, the charged reaction mixture containing dasatinib was added and stirred for 1 hour. The precipitated product was filtered, leaving amorphous dasatinib.

### Example 7—Amorphous Dasatinib

To a reaction flask, charged 2-((6-chloro-2-methylpyrimidin-4-yl)amino)-N-(2-chloro-6-methylphenyl)thiazole-5-carboxamide (20 g), N-(2-hydroxyl ethyl) piperazine (36 g) and n-butanol were added and the reaction mixture was refluxed for 3 hours to 4 hours. The resulting mixture was cooled and charged with phosphoric acid (10 g) and refluxed at a temperature of 50° C. to 60° C. The reaction mixture was stirred for 1 hour at a temperature of 50° C. to 60° C. The mixture obtained was then cooled to 25° C. to 35° C. and stirred for 1 hour maintaining the temperature. The resulting reaction mixture was then filtered and washed with methanol. To the reaction mixture charged water (400 mL) was added and further cooled at a temperature of 15° C. to 20° C. Liquid ammonia (20 mL) was slowly added to the reaction mixture maintaining the temperature at 15° C. to 20° C. The reaction mixture was stirred for 1 hour to isolate the product, amorphous dasatinib. The product was filtered, washed with water and dried under vacuum.

### Example 8—a Crystalline Form C2 of Dasatinib

To a reaction flask, charged dasatinib amorphous form (2 g) and benzyl alcohol (20 mL) were added. The resulting suspension was stirred and maintained at a temperature of 25° C. for 24 to 48 hours to yield a crystalline mixture. The mixture was filtered and dried under vacuum to obtain crystalline Form C2 of dasatinib.

### Example 9—Crystalline Form C3 of Dasatinib

To a reaction flask, charged dasatinib crystalline neat form (N-6) (2 g) and a mixture of benzyl alcohol and methanol (1:1 v/v, 20 mL) were added. The resulting suspension was stirred and maintained at a temperature of 25° C. for 15 to 24 hours to yield a crystalline mixture. The mixture was filtered and dried under vacuum to obtain crystalline Form C3 of dasatinib.

### Example 10—Crystalline Form C3 of Dasatinib

To a reaction flask, charged dasatinib crystalline neat form (N-6) (10 g) and a mixture of benzyl alcohol and ethanol (1:1 v/v, 100 mL) were added. The resulting suspension was stirred and maintained at a temperature of 40° C. for 15 to 24 hours to yield a crystalline mixture. The mixture was then filtered and dried under vacuum to obtain crystalline Form C3 of dasatinib.

### Example 11—Crystalline Form C4 of Dasatinib

To a reaction flask, charged dasatinib crystalline neat form (N-6) (10 g) and a mixture of glycerol and ethanol (1:1 v/v, 100 mL) were added. The resulting suspension was stirred and maintained at a temperature of 25° C. for 15 to 24 hours to yield a crystalline mixture. The mixture was filtered and dried under vacuum to obtain crystalline Form C4 of dasatinib.

1. An amorphous form of dasatinib that exhibits a glass transition onset temperature of 153° C.±3° C.

2. The amorphous form of dasatinib according to claim 1 characterised by a differential scanning calorimetry curve as depicted in FIG. 2.

3. A process for the preparation of an amorphous form of dasatinib according to claim 1, comprising the steps of:
a) mixing dasatinib in a solvent or a mixture of solvents thereof,
b) treating the mixture of step a) with an acid,
c) heating the mixture of step b) to a temperature in the range from about 25° C. to reflux temperature of the solvent used, and
d) treating the mixture of step c) with a base to isolate amorphous dasatinib.

4. The process according to claim 3, wherein the solvent is a polar solvent selected from water, alcohol, acetone or any combination thereof.

5. The process according to claim 4, wherein the alcohol is selected from methanol, ethanol, isopropyl alcohol, n-butanol and 2-butanol or any combination thereof.

6. The process according to claim 3, wherein the acid is an inorganic acid or an organic acid.

7. The process according to claim 6, wherein the inorganic acid is selected from hydrochloric acid, boric acid and phosphoric acid or any combination thereof.

8. The process according to claim 6, wherein the organic acid is selected from formic acid, acetic acid, oxalic acid, citric acid, succinic acid, benzoic acid and p-toluenesulfonic acid or any combination thereof.

9. The process according to claim 3, wherein the mixture in step c) is heated to a temperature of about 50° C. to about 95° C.

10. The process according to claim 3, wherein the mixture is cooled to a temperature of about 15° C. to about 45° C.

11. The method of claim 3, wherein the product from step (c) is isolated as a solid before being treated with a base in step (d).

12. The process according to claim 3, wherein the base is an inorganic base or an organic base selected from ammonia, triethylamine, potassium carbonate, potassium bicarbonate, sodium carbonate and sodium bicarbonate or any combination thereof.

13. The process according to claim 3, wherein the mixture is maintained at a temperature of about 5° C. to about 30° C., for a period of about 0.5 hour to about 3 hours.

14. A process for the preparation of an amorphous form of dasatinib according to claim 1 comprising the steps of:
a) mixing dasatinib with a solvent or a mixture of solvents thereof,
b) heating the reaction mixture at a temperature of 50° C. to 90° C.,
c) treating the reaction mixture with at least one water miscible solvent or a mixture thereof, and
d) isolating the amorphous form of dasatinib.

15. The process according to claim 14, wherein the solvent used in step a) is a polar solvent selected from water, alcohol, acetone or any combination thereof.

16. The process according to claim 15, wherein the alcohol is selected from methanol, ethanol, isopropyl alcohol, n-butanol and 2-butanol or any combination thereof.

17. The process according to claim 14, wherein the water miscible solvent used in step c) is selected from acetonitrile, acetone, DMF, DMSO and THF or any combination thereof.

18-32. (canceled)

33. A pharmaceutical composition comprising dasatinib according to claim 1.

34. The pharmaceutical composition comprising dasatinib according to claim 33, in combination with one or more pharmaceutically acceptable excipients.

35. A method comprising utilizing the dasatinib according to claim 1, or of the pharmaceutical composition comprising dasatinib according to claim 1 in treating cancer.

36. A method for treating cancer comprising administering to a patient in need thereof a therapeutically effective amount of dasatinib according to claim 1, or of the pharmaceutical composition comprising dasatinib according to claim 1.
